(19) United States
(12) Patent Application Publication
(10) Pub. No.: US 2017/0035624 A1
(43) Pub. Date: Feb. 9, 2017

(54) FEMININE HYGIENE ARTICLE

(71) Applicant: The Procter & Gamble Company, Cincinnati, OH (US)

(72) Inventors: Ivano Antonio GAGLIARDI, Pescara (IT); Sabine MEISL, Muehlheim (DE)

(21) Appl. No.: 14/819,876

(22) Filed: Aug. 6, 2015

(51) Int. Cl. A61F 13/472

(52) U.S. Cl. CPC A61F 13/47236

(57) ABSTRACT

A feminine hygiene article comprises a topsheet, a backsheet and an absorbent core disposed therebetween. The feminine hygiene article has two longitudinal side edges. Each longitudinal side edge has a notch that extends from the longitudinal side edge towards the absorbent core. Each notch is disposed between a front portion transverse centerline and rear portion transverse centerline of the feminine hygiene article.

### FIELD OF THE INVENTION

The present invention relates to feminine hygiene articles, such as sanitary napkins, adult incontinence articles, pantiliners and the like.

### BACKGROUND OF THE INVENTION

Feminine hygiene articles are typically articles which are placed by a consumer in her undergarment and comprise articles commonly referred to as sanitary napkins (or sanitary pads), adult incontinence absorbent articles, pantiliners, and the like. Whereas sanitary napkins are generally used to collect relatively large flows of liquid (menses), pantiliners are relatively thin feminine hygiene articles designed to protect the user's underwear and clothing from soiling by light vaginal discharges. For example, many women have developed the habit of wearing a pantiliner between their menstrual periods to protect their clothing from bodily fluids such as vaginal discharges and slight urinary leak.

Currently, feminine hygiene articles are typically sold in packages that contain a number of individual articles, usually all of the same size and shape. With respect to the undergarments in which such feminine hygiene articles are used, there are many different styles and cuts of undergarments, such as briefs, bikini, thong, french cut, hipster, and the like. As such, the feminine hygiene article needs to be compatible with a variety of styles of undergarments. A feminine hygiene article that may provide suitable comfort, fit and protection when used in one style of undergarment, may be deficient when used in another style of undergarment.

For example, typical pantiliners are designed for use with undergarment styles having a full-sized crotch portion, such as briefs and bikini styles. However, such pantiliners do not readily lend themselves for use with undergarments having a narrower crotch portion, such as thong style undergarments. When typical pantiliners are utilized in thong style undergarments, the consumer will fold and wrap the sides of the article at the rear portion of the article around the crotch and rear portion of the thong undergarment. As a result, the pantiliner can become uncomfortably thick and have a sharp edge at the point in which the article is folded by the consumer, resulting in an uncomfortable experience.

There thus remains a desire to develop a feminine hygiene article that provides suitable comfort, fit and protection not only in undergarments will full-size crotch portions, but also in undergarments with narrower crotch portions such as thong style undergarments.

### SUMMARY OF THE INVENTION

The present invention relates to a feminine hygiene article comprising a topsheet, a backsheet, and an absorbent core disposed between the topsheet and backsheet. The absorbent core has a front longitudinal core end and a rear longitudinal core end. The absorbent core has a core transverse centerline between the front longitudinal core end and the rear longitudinal core end, and a core rear portion transverse centerline between the core transverse centerline and the rear longitudinal core end. The absorbent core has a first core width at the core transverse centerline which is greater than a second core width at the core rear portion transverse centerline.

The feminine hygiene article has a first longitudinal article side edge, a second longitudinal article side edge, a front longitudinal article end, a rear longitudinal article end, an article transverse centerline, an article front portion transverse centerline between the article transverse centerline and the front longitudinal article end, and an article rear portion transverse centerline between the article transverse centerline and the rear longitudinal article end.

The feminine hygiene article further comprises a first notch that extends from the first longitudinal article side edge towards the absorbent core and a second notch extending from the second longitudinal article side edge towards said absorbent core. Each notch is disposed between the article front portion transverse centerline and the article rear portion transverse centerline.

The notches in the longitudinal side edges of the article allow a consumer to fold and wrap the sides of the rear portion of the article around the undergarment without creating unnecessary thickness and sharp edges at the point in which the article is folded by the consumer. This provides a much more comfortable and acceptable wearing experience for the consumer.

### BRIEF DESCRIPTION OF THE DRAWINGS

FIG. 1 is a top view of a feminine hygiene article of the invention.

FIG. 2 is a top view of the feminine hygiene article of FIG. 1 placed in a thong style undergarment.

FIG. 3 is a top view of a feminine hygiene article of the invention.

### DETAILED DESCRIPTION OF THE INVENTION

The feminine hygiene article of the present invention comprises a topsheet, a backsheet and an absorbent core disposed therebetween. The backsheet has a body-facing side oriented towards the body of the wearer of the article and a garment-facing side oriented towards the undergarment of the wearer of the article. The feminine hygiene article has two longitudinal article side edges, each of which have a notch extending from the longitudinal side edge towards the absorbent core.

The feminine hygiene articles of the present invention are generally articles which are placed by a consumer in her undergarment and comprise articles commonly referred to as sanitary napkins (or sanitary pads), adult incontinence absorbent articles, pantiliners, and the like.

### Topsheet

The topsheet is typically the layer of the feminine hygiene article which is oriented towards and contacts the body of the wearer, and is therefore the first layer to receive the bodily discharges. The topsheet is normally made of a single layer, but may also comprises more than one layer (for example a central topsheet layer and two overlapping lateral stripes, as disclosed in WO93/09744 or EP766,953).

The topsheet is normally liquid pervious. The term “liquid pervious” as used herein refers to components that allow liquids to pass therethrough without significantly retarding or obstructing the transmission of such liquids therethrough.

It is envisaged that any conventional topsheet materials may be used within the invention. Advantageously, the topsheet may not be opaque so that a visual indicator may be disposed on a layer underlying the topsheet and still be visible by a user looking upon the body-facing side of the topsheet 12. Suitable topsheets may be made for example from nonwoven materials or perforated polyolefinic films. An exemplary topsheet suitable for use herein is a relatively hydrophobic 20 gsm spunbonded nonwoven web comprising bicomponent fibers of the sheath core type (PP/PE), exemplarily available from Pegas a.s., Czech Republic.

If desired, the topsheet may be treated with a surfactant to enhance liquid penetration to the core. The surfactant is typically non-ionic and should be nonirritating to the skin. A surfactant density of about 0.01 milligrams per square centimeter of topsheet area is normally suitable. An exemplary surfactant is sold by the Glyco Chemical, Inc. of Greenwich, Connecticut as Pegosperse 200 ML. The topsheet may have a plurality of apertures to permit liquids deposited thereon to pass through to the core more quickly.

The topsheet and the backsheet are preferentially peripherally joined using known techniques such as heat embossing. The layers may also be glued to each other. The topsheet may be contiguous with the backsheet with these two layers forming the outer periphery of the article.

### Absorbent Core

The feminine hygiene articles of the invention further comprise an absorbent core disposed between the topsheet and the backsheet. As used herein, the term “absorbent core” refers to a material or combination of materials suitable for absorbing, distributing, and storing fluids such as urine, blood, menses, and/or other body exudates.

The size and shape of the absorbent core may be such that the surface of the core in the horizontal plane is substantially smaller than the surface of the topsheet. By “substantially smaller”, it is meant that the surface of the absorbent core is at least about 10% smaller than the surface of the topsheet, or at least about 25% smaller than the surface of the topsheet. The absorbent core may be generally centered in the middle of the article. The absorbent core may be disposed away from the periphery of the article to provide improved flexibility along the edges of the article.

By providing an absorbent core having a substantially smaller surface than the topsheet, several benefits may be achieved. The amount of core material used is reduced, lowering the overall costs of manufacturing the product. A core having a smaller surface also increases the overall flexibility of the product, because the regions of the product not provided with a core are generally less rigid than the region where the core is situated.

The absorbent core of the present invention has a front longitudinal core end, a rear longitudinal core end, a core transverse centerline between the front longitudinal core end and the rear longitudinal core end, and a core rear portion transverse centerline between the core transverse centerline and the rear longitudinal core end. The absorbent core will have a first core width at said core transverse centerline which is greater than a second core width at said core rear portion transverse centerline. As such, the absorbent core can have a width that tapers (i.e. decreases) from the center of the core towards the rear end of the core.

The absorbent core can have an oval shape as represented in the figures but may also have any other shape. For example it is typical for absorbent cores to be rectangularly shaped for ease of manufacturing. However, flexibility and compatibility with various styles of undergarments may be better with cores having a curved shape (such as an oval shape) and not comprising right angles.

The absorbent core can be made of any suitable materials. Nonlimiting examples of suitable liquid-absorbent materials include comminuted wood pulp which is generally referred to as airfelt; creped cellulose wadding; absorbent gelling materials including superabsorbent polymers such as hydrogel-forming polymeric gelling agents; chemically stiffened, modified, or cross-linked cellulose fibers; meltblown polymers including co-form; synthetic fibers including crimped polyester fibers; tissue including tissue wraps and tissue laminates; capillary channel fibers; absorbent foams; absorbent sponges; synthetic staple fibers; peat moss; or any equivalent material; or combinations thereof. The absorbent core can comprise superabsorbent polymer (SAP), normally distributed within a matrix of cellulosic fibers, for example in order to reduce the thickness of the absorbent core.

The absorbent core can be unitary, or can be a laminate of two or more layers. For example, the core can comprise a fluid impermeable barrier layer (e.g. a PE Patch) on its backsheet-facing side to prevent fluids retained by the absorbent core from striking through the pantiliner and soiling adjacent garments. An exemplary PE patch is a 25 gsm poly film available from Britton Taco (UK) under trade name ST-012A-White. Further general information regarding absorbent cores can be found in prior patent publications, see for example WO0207662A1 and WO9119471.

### Backsheet

The general function of the backsheet is to prevent discharges absorbed by the core from escaping the feminine hygiene article and soiling the clothing of the wearer. The backsheet may be made of any suitable material in particular any standard backsheet materials. These materials are generally flexible, liquid resistant, and liquid impervious.

Any conventional backsheet materials may be used within the invention, such as polyolefinic films or nonwoven webs. Nonwoven webs may be advantageous because they normally provide better breathability for the articles and may be cheaper than polyolefinic films. For example, a relatively hydrophobic 18 grams per square meter (gsm) spunbonded nonwoven web of 2 denier polypropylene fibers, exemplarily available from Pegas a.s. Czech Republic. The backsheet may also be a laminate as is know in the art.

The backsheet has a garment-facing side and an opposite body-facing side. The garment-facing side of the backsheet can comprise a non-adhesive area and an adhesive area. The adhesive area may be provided by any conventional means. Pressure sensitive adhesives have been commonly found to work well for this purpose.

Full adhesive coverage of the backsheet may be applied except for a relatively small non-adhesive area at the front end portion of the article serving as finger lift. It is known that providing a full adhesive coverage of the garment-facing side of the backsheet except for a non adhesive area may be advantageous to provide a better adherence of the article to the undergarment. It can be preferred to have full adhesive coverage on the garment-facing side of the backsheet between the article transverse centerline and article rear end of the article, which enables the longitudinal side edges of the article towards the rear portion to be wrapped around and adhered to the clothes-facing side of the undergarment in which the article is placed.

The shape of the adhesive area may also be different, for example it is also common to use one or two or more strips of longitudinally oriented adhesive strips instead of full coverage. The adhesive strips may then be continuous or intermittent. For example two longitudinally oriented strips, one on each side of the longitudinal axis, may be applied. The adhesive may be applied via direct slot coating application process.

Mechanical adhesive means may be also provided, such as microscopic hooks placed on the backsheet and designed to attach to the fibers present in some undergarments, as in a hook-and-loop fastener.

### Notches

The feminine hygiene article of the present invention importantly comprises a notch along each longitudinal side edge of the article. These notches enable the consumer to conveniently fold and wrap the sides of the rear portion of the article around an undergarment, especially a thong style undergarment, without creating unwanted thickness and sharp edges at the point in which the article is folded. The notches therefore improve the comfort of the article in use.

Each longitudinal side edge of the article comprises at least one notch. In one aspect, each longitudinal side edge of the article has only one notch, preferably with each notch disposed along the same transverse axis of the article. In another aspect, each longitudinal side edge of the article can comprise two or more notches, in order to facilitate adaptation to various styles of undergarments.

The notches are generally a portion of the longitudinal side edges of the article which have been cut out or slitted, typically during manufacture of the article. The notches can therefore be formed by removing topsheet material and backsheet material, such that each notch is free of topsheet material and backsheet material. Notches can also be pre-formed in the topsheet and/or backsheet materials of the article. The notches can be in a variety of shapes, such as rounded-V-shaped notches or simple slits. The notches extend from each longitudinal edge of the article towards the absorbent core. The notches preferably do not extend all the way to the absorbent core, as this could give rise to a risk of leakage of absorbed bodily fluid. Furthermore, the notches preferably do not impinge, or otherwise disrupt or alter the shape of, the outer perimeter of the absorbent core.

Each notch is positioned between the article front portion transverse centerline and said article rear portion transverse centerline in order to effectively facilitate the folding and wrapping of the rear portion of the article around the undergarment, especially a thong style undergarment.

### Release Cover

The feminine hygiene article preferably will further comprise a release cover releasably attached to an adhesive area of the garment-facing side of the backsheet. The release cover is removed by the user before placing the article in the undergarment. Different types of release covers exist, in particular so-called release paper, one-piece wrapper sheet and two-piece wrapper sheet. Any conventional release covers may be used with the present invention.

Release papers are normally used for articles that are provided flat (not folded) to the user and may be of about the same size (or smaller) as the article. The release papers may be made of a paper material, one side of which has been treated with silicone and this siliconized layer may be releasably attached to the adhesive area. Sometimes a non-woven material is used in place of a paper material, but the principle and use remains the same. The term release paper has used herein will therefore refer to all release covers which are not a wrapper sheet, in particular release covers used for articles sold in a non-folded configuration and/or which do not extend beyond the periphery of the article. The release paper may also comprise a small cut-out section in an area corresponding to the non-adhesive area, in order to further help the removal of the release paper from the backsheet.

These various types of release covers are known in the art. Suitable examples include BL 30 MG-A SILOX EI/O, BL 30 MG-A SILOX 4 P/O available from Akrosil Corporation, and M&W films available from Gronau in Germany.

The thickness of the feminine hygiene articles according to the invention may be typical for the type of applications foreseen. For pantiliners (as exemplarily represented in the Figures), the thickness is generally thinner as compared to sanitary pads. The articles of the invention may have a relatively small thickness (also called “caliper”) of less than about 5 millimeters, or less than about 3 millimeters, as measured using the standard test described below. The articles may be even thinner, to provide very discrete articles. The lowest limit for the thickness of the article will be dictated by technical feasibility, but obtaining an effective article thinner than 0.4 mm may be difficult. Suitable thickness ranges for the articles of the invention include from about 0.4 mm to about 4 mm, and from about 0.6 mm to about 3 mm. As used herein, the term “thickness of the article” refers to the thickness value measured in the center of the article, i.e. normally including the thickness of the core.

The dimensions of the articles of the invention in the horizontal plane are typical of the articles in the field. For example, the length of such products will normally lie in a range of from about 8 cm to about 20 cm for the length of the article, and of from about 3 to about 9 cm for its width. The overall surface of the article (so typically the surface of the topsheet) may also lie within the usual range found for these articles, which normally would be from about 40 cm² to about 250 cm². For the purpose of providing exemplary dimensions of a pantiliner such as the one represented in the Figures, such a pantiliner may have a length of 15 cm, a width (at center) of 4.8 cm, an overall surface of the article of 79 cm² and a core area surface of 38 cm². An exemplary thickness of the article represented is about 1 mm.

The feminine hygiene article is preferably free of side panels, otherwise known as “wings”, which extend from the transverse centerline area of the article and are intended to be wrapped around the undergarment and attached to the clothes-facing side of the undergarment. As such the feminine hygiene article can have a width at the article transverse centerline that is less than the width at the article front portion transverse centerline and less than the width at the article rear portion transverse centerline.

The feminine hygiene article can further comprise fold lines (e.g. embossments) which help to conform the article to the undergarment, such as the fold lines described in EP 1260205.

The feminine hygiene article can further comprise a colored area, for example by printing ink, to highlight the location of the notches of the article. The area of the article adjacent the notches can therefore be colored. This can also help to facilitate proper placement of the article in the undergarment by the consumer.

The articles of the invention are normally disposable, i.e. are not intended to be re-usable or washable, and are normally disposed of after a single use.

### Method of Manufacture

The sanitary articles of the present invention may be produced industrially by any conventional means. The different layers may thus be assembled using standard means such as embossing (e.g. thermal bonding) or gluing or a combination of both. The converting line may comprise a printing step wherein the ink is applied to the backsheet of the article. It is however also possible to carry the printed step on the backsheet outside the converting line of the article, before this layer is joined with any of the other layers.

### Method of Use

The present invention further relates to a method of absorbing bodily fluids (such as menses, urine, blood, or the like) comprising the steps of placing the feminine hygiene article of the present invention in an undergarment, preferably a thong style undergarment, and contacting the feminine hygiene article with bodily fluid.

### Thickness Measurement

The articles of the inventions may be relatively thin and not bulky, so that the thickness measurements will be less dependent of the pressure applied when making the measurement, than for example for bulky articles such as thick pads. The following method can be used to measure the thickness of the article of the invention. The equipment can comprise an apparatus capable of measuring thickness with a 0.05 mm tolerance. An example of such equipment is Mitutoyo Micrometer Gauge having a range of 0-30 mm The micrometer is not spring-loaded (i.e. the foot only moves downward under gravity). The caliper gauge is fitted with a circular foot made of aluminum, which has a 40 mm contact foot diameter. A stainless steel weight of 80 grams is placed on the spindle extending above the micrometer dial.

The test procedure is as follows. Make sure the micrometer is zeroed. Place the article without the release cover on the base plate, the topsheet facing up. If the article was provided in a compressed state (as is sometimes the case in certain packaging), the article is let to rest about 5 minutes before its thickness is measured. Similarly, if the article was provided folded, the article is first opened and let about 5 minutes to rest in its “flat” shape. Position the article on the base plate so that when the foot is lowered, it is in the center of the article. Let the foot gently lowers itself onto the article. Determine the article caliper by reading the micrometer dial between 5 seconds and 10 seconds after the foot comes in contact with the article.

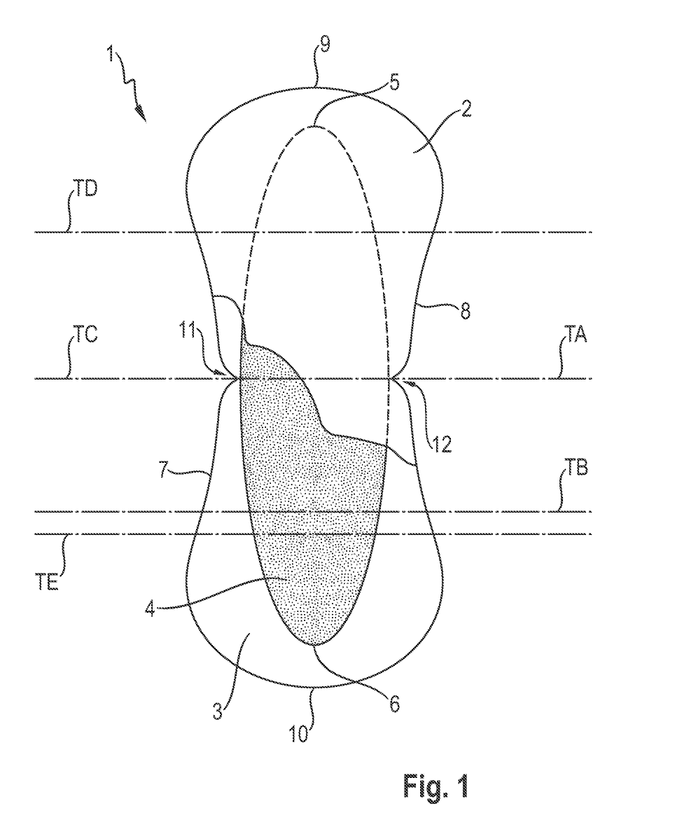

Referring now to the Figures, FIG. 1 illustrates a feminine hygiene article 1 of the present invention comprising a topsheet 2, a backsheet 3, and an absorbent core 4 disposed between the topsheet 2 and the backsheet 3. The article 1 is cut-away to show the absorbent core 4 and backsheet 3. The absorbent core 4 has a front core end 5, a rear core end 6, a core transverse centerline (TA) between the front core end 5 and the rear core end 6, and a core rear portion transverse centerline (TB) between the core transverse centerline (TA) and the rear core end 6. The absorbent core 4 has a first core width measured at the core transverse centerline (TA) which is greater than a second core width measured at the core rear portion transverse centerline (TB). The absorbent core 4 further has a third width at the rear core end 6 that is less than the second width of the absorbent core 4 at the core rear portion transverse centerline (TB). As such, the width of the absorbent core decreases from the core transverse centerline to the rear core end 6 (in a linear or curvilinear fashion).

The feminine hygiene article 1 of FIG. 1 has a first longitudinal article side edge 7, a second longitudinal article side edge 8, a front article end 9, a rear article end 10, an article transverse centerline (TC), an article front portion transverse centerline (TD) between the article transverse centerline (TC) and the front article end 9, and an article rear portion transverse centerline (TE) between the article transverse centerline (TC) and the rear article end 10. The width of the feminine hygiene article 1 at the article transverse centerline (TC) is less than the width of the feminine hygiene article 1 at the article front portion transverse centerline (TD) and at the article rear portion transverse centerline (TE).

The feminine hygiene article 1 of FIG. 1 further comprises a first notch 11 extending from the first longitudinal article side edge 7 towards the absorbent core 4 and a second notch 12 extending from the second longitudinal article side edge 8 towards the absorbent core 4, each notch 11, 12 being disposed between the article front portion transverse centerline (TD) and the article rear portion transverse centerline (TE). Each of the notches 11, 12 have a rounded-V shape.

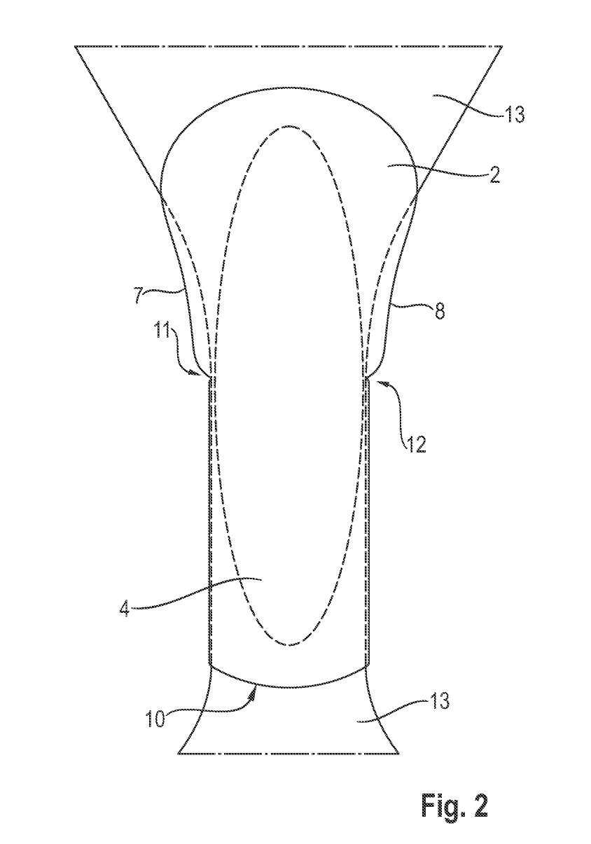

FIG. 2 illustrates the feminine hygiene article 1 of FIG. 1 after having been placed in a thong style undergarment 13 by a consumer. As can be seen in FIG. 2, the notches 11, 12 disposed along each longitudinal side edge 7, 8 of the article 1 allow the longitudinal sides 7, 8 between the notches and the rear article end 10 to be more easily folded and wrapped around the undergarment without creating unnecessary thickness or sharp edges at the point where the notches are located. This allows a much more comfortable experience for the wearer of the article during use.

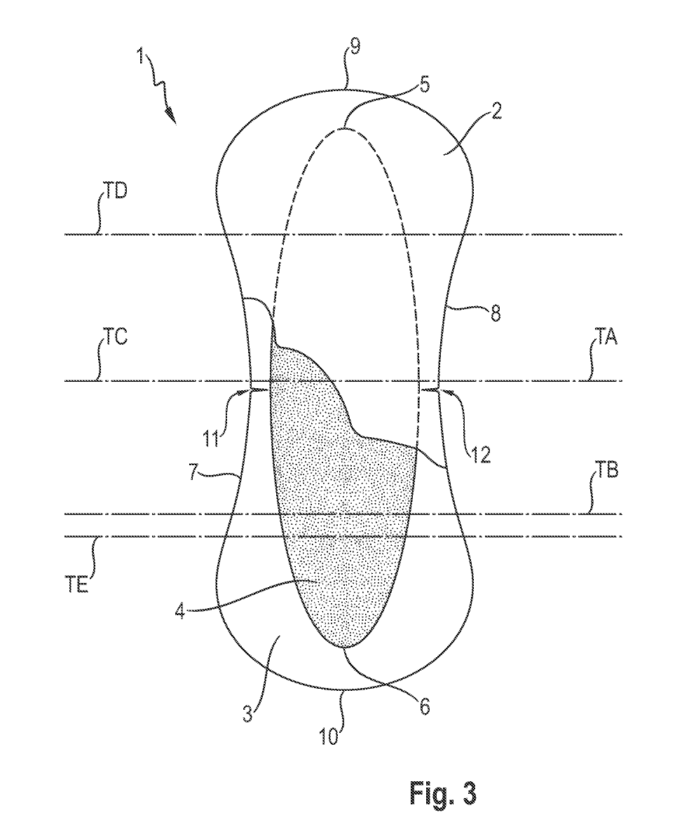

FIG. 3 illustrates another feminine hygiene article 1 of the present invention having the features as shown in FIG. 1, except that the first and second notches 11, 12 are in the form of slits. While this notch design can be less preferred as compared to the rounded-V-shaped notch of FIG. 1, it nonetheless allows the article to be more easily folded and wrapped around the undergarment without creating unnecessary thickness at the point in which the article is folded and wrapped around the undergarment, making the article more comfortable, especially when worn in thong style undergarments. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A feminine hygiene article comprising:
a topsheet;
a backsheet; and
an absorbent core disposed between said topsheet and said backsheet, said absorbent core having a front core end, a rear core end, a core transverse centerline between said front core end and said rear core end, and a core rear portion transverse centerline between said core transverse centerline and said rear core end; wherein a first core width of said absorbent core at said core transverse centerline is greater than a second core width of said absorbent core at said core rear portion transverse centerline;
wherein said feminine hygiene article has a first longitudinal article side edge, a second longitudinal article side edge, a front article end, a rear article end, an article transverse centerline, an article front portion transverse centerline between said article transverse centerline and said front article end, and an article rear portion transverse centerline between said article transverse centerline and said rear article end; and
wherein said feminine hygiene article comprises a first notch extending from said first longitudinal article side edge towards said absorbent core and a second notch extending from said second longitudinal article side edge towards said absorbent core, each notch disposed between said article front portion transverse centerline and said article rear portion transverse centerline.

2. The feminine hygiene article of claim 1, wherein a third core width of said absorbent core at said rear longitudinal core end is less than said second width of said absorbent core at said core rear portion transverse centerline.

3. The feminine hygiene article of claim 1, wherein a width of said feminine hygiene article at said article transverse centerline is less than a width of said feminine hygiene article at said article front portion transverse centerline and/or at said article rear portion transverse centerline.

4. The feminine hygiene article of claim 1, wherein said feminine hygiene article has a thickness of less than about 5 millimeters, preferably less than about 3 millimeters.

5. The feminine hygiene article of claim 1, wherein said first notch and said second notch are each rounded-V-shaped.

6. The feminine hygiene article of claim 1, wherein said first notch and said second notch are each slits.

7. The feminine hygiene article of claim 1, wherein the surface of the absorbent core is at least about 10% smaller than the surface of the topsheet.

8. The feminine hygiene article of claim 1, wherein said absorbent core is oval-shaped.

9. The feminine hygiene article of claim 1, wherein said backsheet has a garment-facing side and a body-facing side, wherein said garment-facing side of said backsheet has adhesive applied thereto, wherein said feminine hygiene article further comprises a release cover releasably attached to said backsheet by said adhesive.

10. The feminine hygiene article of claim 1, wherein each notch is formed by removing a portion of topsheet material and backsheet material, and wherein each notch is free of topsheet material and backsheet material.

11. The feminine hygiene article of claim 1, wherein each notch does not impinge on the outer perimeter of the absorbent core.

12. A method of absorbing bodily fluid comprising the steps of placing a feminine hygiene article according to any one of the preceding claims in an undergarment and contacting said feminine hygiene article with bodily fluid.

13. The method of claim 12, wherein said undergarment is a thong style undergarment.
